(19) United States
(12) Patent Application Publication
(10) Pub. No.: US 2005/0075555 A1
(43) Pub. Date: Apr. 7, 2005

(54) System and method for in vivo sensing

(76) Inventors: Arkady Glukhovsky, Santa Clarita, CA (US); Yoram Palti, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/496,944

(22) PCT Filed:
(86) PCT No.: PCT/IL03/00376

Related U.S. Application Data
(60) Provisional application No. 60/378,684, filed on May 9, 2002.

(51) Int. Cl. A61B 5/05; H04N 13/00

(52) U.S. Cl. 600/407; 348/48; 348/77

(57) ABSTRACT

An apparatus, system and method that enables sensing and/or measuring environmental conditions in an in vivo environment. An environment sensitive material, such as a temperature sensitive color changing material, may be placed within or without of an in-vivo imaging device. The environment sensitive material may change in response to environmental changes, such as temperature changes, pH level changes, pressure changes etc., and the in-vivo imaging device may acquire these responses. These responses may be acquired by an imager, in the form of images that indicate the color status of the environment sensitive material for each data frame sent from the in-vivo imaging device to a data receiving unit and/or data processor. The data may be processed and analyzed etc. by a data processor, and output by an output device.

### FIELD OF THE INVENTION

The present invention relates to systems, methods, and apparatuses useful in sensing and/or measuring in vivo conditions. Specifically, embodiments of the present invention relate to at least one apparatus, system, and method that provide for sensing or measuring of temperature, pressure and pH levels etc. in in-vivo environments.

### BACKGROUND OF THE INVENTION

In many circumstances it may be important to measure in vivo conditions, such as temperature, pressure or pH levels etc. inside a body. Such circumstances may occur, for example, during medical diagnostics and/or treatment of internal parts of a body.

In living bodies, parameters such as temperature, pressure and/or pH changes etc. can be indicative of a pathology or abnormality etc. It may be important to measure in vivo parameters and optionally attain real time feedback as to the parameters. Furthermore, it may be important to be able to measure and optionally provide real time feedback for in vivo parameters that are typically difficult to access for conventional measuring mechanisms. For example, it is typically difficult to provide instrumentation that may access an area such as the gastrointestinal (GI) tract. Of course, other structures and areas of the body may require such sensing or measuring.

It would be highly advantageous to have a measuring means that may reach places within the body that are usually difficult to reach, and provide in vivo data for environmental parameters, optionally providing real time feedback of this data.

### SUMMARY OF THE INVENTION

There is provided, in accordance with an embodiment of the present invention, an apparatus, system, and method for sensing an environment, such as inside a body (in vivo). The apparatus, system, and method may utilize, for example, temperature and/or pH and/or pressure sensitive color-changing material etc. to indicate internal body environmental changes. In one embodiment, the material that may be used is thermotropic liquid crystal.

According to some embodiments, an apparatus may include an ingestible device, such as a swallowable capsule, having color-changing material placed on an inner and/or outer surface. According to some embodiments of the present invention, there may be at least one light source for illuminating the color-changing material, and an imager capable of capturing images of the color-changing material. According to some embodiments of the present invention, environmental temperature (and/or pH and/or pressure etc.) and/or a change of environmental temperature (and/or pH and/or pressure etc.) may result in a color change of the environment sensitive color changing material. Samples of acquired data that indicate the color status of the environment sensitive color changing material may be transmitted, for example, to a data receiving unit, stored in a storage unit, processed by a processing unit and/or displayed by an output device, optionally in real time. The color change of the material may typically be determined according to the relation between each color and the measured environment parameter or value (temperature, pressure, pH, etc).

### BRIEF DESCRIPTION OF THE DRAWINGS

The principles and operation of the system, apparatus, and method according to embodiments of the present invention may be better understood with reference to the drawings, and the following description, it being understood that these drawings are given for illustrative purposes only and are not meant to be limiting, wherein:

FIG. 1 is a schematic illustration of various components of a device and viewing system;

FIG. 2 is a schematic illustration of a measuring sensitive element placed within an in-vivo device, according to some embodiments of the present invention;

FIG. 3 is a is a block diagram illustration of a work flow between various system components, according to some embodiments of the present invention;

FIG. 4 is a flowchart illustrating a method of measuring in vivo temperature changes, according to some embodiments of the present invention;

FIG. 5 is a calibration curve illustrating a relationship between hue values and temperature for a measurement material, according to some embodiments of the present invention;

FIG. 6 is a calibration curve illustrating a second relationship between hue values and temperature for a measurement material, according to some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements throughout the serial views.

### DETAILED DESCRIPTION OF THE INVENTION

The following description is presented to enable one of ordinary skill in the art to make and use embodiments of the present invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

The platforms, processes and displays presented herein are not inherently related to any particular computer or other apparatus. Various general-purpose computing systems and networking equipment may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the present invention as described herein.

In the following detailed description, numerous specific details are set forth in order to provide a thorough understanding of the present invention However, it will be understood by those skilled in the art that embodiments of the present invention may be practiced without these specific details.

It will be appreciated that the term “environment” in the present invention relates to any space in which an in-vivo device may function, including, for example, within body lumen, cavity, organ, canal, wall etc. The phrase “environmental parameters” as used hereinafter may encompass, for example, temperature levels, pH levels, pressure levels, bacteria levels or any other relevant in vivo parameters that may be measured The phrase “in vivo” as used hereinafter may encompass any space within a living organism, such as inside a body of a living organism, including a human body, animal body, or any other suitable body.

It is noted that while the embodiments of the invention shown hereinbelow are typically adapted for imaging of the gastrointestinal (GI) tract, the devices and methods disclosed herein may be adapted for imaging other body cavities or spaces etc.

Specifically, embodiments of the present invention enable sensing or measuring of in vivo environmental parameters, and optionally enabling analysis and display of these parameters or parameter changes. According to some embodiments of the present invention, measuring material or elements may be placed within or on an in-vivo device. Data attained by these elements may be transmitted to a data receiving unit and to a data processor and image monitor etc.

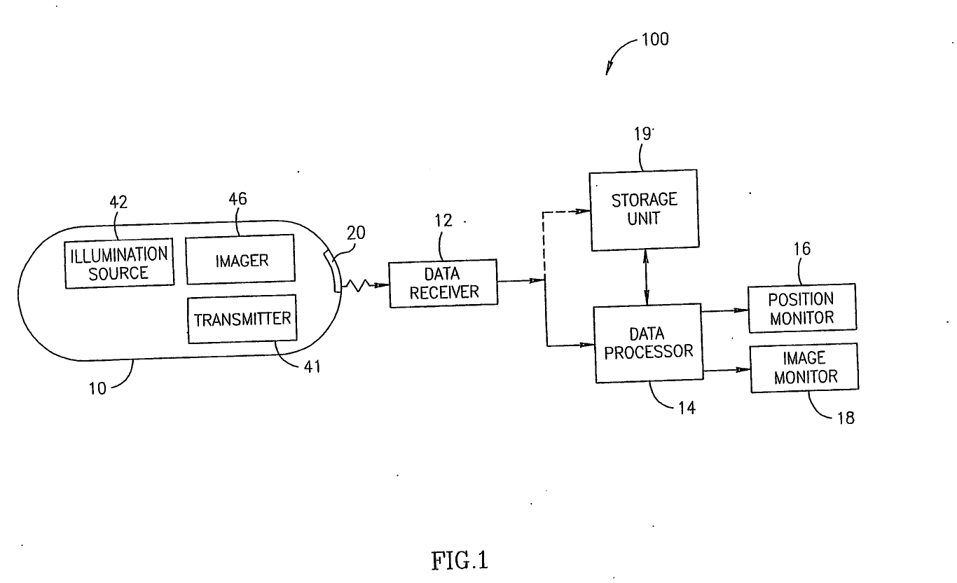

Reference is now made to FIG. 1, which is a schematic illustration of an in-vivo imaging device and system 100, according to some embodiments of the present invention. Such an embodiment may include, for example: a swallowable capsule 10, with an imager 46, transmitter 41, and environment sensing element 20; a data receiver unit 12 for receiving in-vivo imaging device data; a data processor 14; and displaying apparatuses such as 16 and 18. For example, a data receiver unit 12 may receive the data from the in-vivo imaging device 10, and may thereafter transfer the data to a data processor 14, and optionally a data storage unit 19. The data may be displayed on a position monitor 16 and/or an image monitor 18. While FIG. 1 shows separate monitors, both an image and its position can be presented on a single monitor. Data receiver unit 12 may be separate from the processing unit 14 or combined with it. Data processor 14 may be, for example, a personal computer or workstation, and may include, for example, a processor memory etc. Data processor 14 may be configured for real time processing and/or for post processing to be viewed or otherwise displayed at a later date. Units 14, 16, 18 and 19 may be integrated into a single unit, or any combinations of the various units may be implemented. Of course, other suitable components may be used.

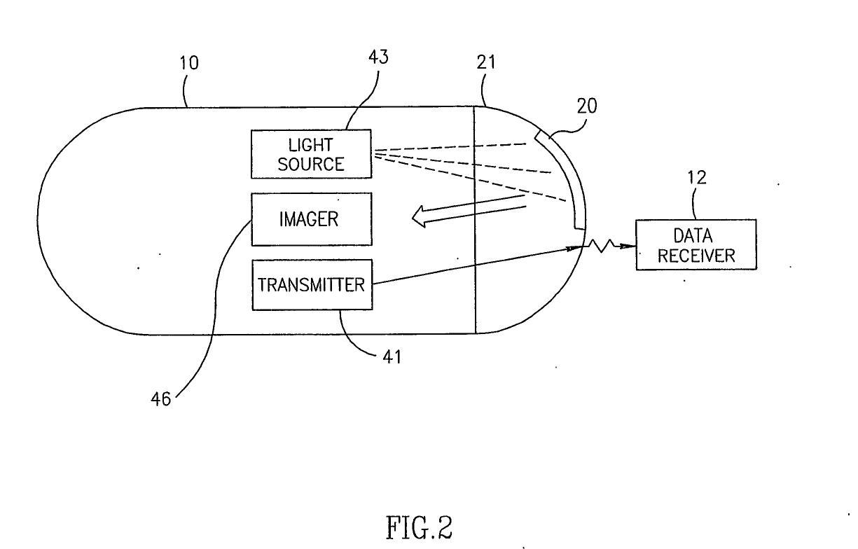

Detection of various environmental parameters, such as internal temperature, pressure and pH levels etc. may be enabled by determining, for example, the changes in color of parameter sensitive color-changing materials, such as temperature sensitive material. Other environment sensors or environmental parameter sensitive materials etc. may be used to determine alternative internal environmental changes. Reference is now made to FIG. 2, which is a schematic diagram of a color changing material 20 attached to an in-vivo imaging device 10. An example of a temperature sensitive color changing material is a thermotropic color changing liquid crystal material. An example of pH sensitive color changing material is litnus paper. Another example of pH sensitive color changing material is Liquid crystal material that may change color in response to pressure. Examples of pressure sensitive color changing materials are shear-sensitive liquid crystal coatings (SSLCC) that when applied to planar surfaces may reveal (via color change) the nature of a shearing force. It is known that changes in applied shear stress magnitude may cause the liquid crystal molecular arrangement to change, thereby reorienting the scattered light spectrum in space. A fixed observer may thereby see color change in response to the altered shearing force. Such color changes may be continuous and reversible, with time response in the order of milliseconds. Another example of a pressure sensitive color changing material is a “Pressurex”, a color changing irreversible material by Sensor Products Inc. (188 Rt. 10 Suite 307 East Hanover, N.J. 07936-2108 USA).

The in-vivo device 10 may record or otherwise acquire images of the color changing material 20, using at least imager 46. Imager 46 may also image an in vivo site. The acquired images may be transmitted to a data receiving unit 12 and/or storage unit 19 and/or data processor 14. The data from the acquired images of the color changing material 20, for example, may be processed, analyzed and/or viewed etc. on, for example, a position monitor 16 and/or image monitor 18. The data may be presented as a number, as a graph, as a color chart or map, or in any other form. Optionally, the color-changing material 20 may be viewed while inside the body lumen so that any color change of the material may be detected, analyzed and/or presented to a viewer, typically a doctor, optionally in real time. The detection and/or analysis and/or display etc. of the acquired image data may include translation of the data into a corresponding change of the measured parameter. For example, the detection and display of an in vivo temperature change may be indicated by a corresponding change in color of the color changing material 20.

In addition to revealing pathological conditions of body lumen, some embodiments system 100 may provide information about the location of these pathologies, for example in the gastrointestinal tract (GI) tract. The information obtained by visual means, by viewing the color changing material 20 or information obtained by processing such color information, may be complemented and/or localized by providing information relating to alternative local (environmental) conditions, such as pH and/or pressure levels etc. in, for example, the GI tract or other body lumens, such as the reproductive tract etc. In this way an in-vivo imaging device may provide data for a frame that includes more than one environmental parameter, such as, for example, temperature and pH level in an environment etc. For example, by placing a plurality of different sensitive materials 20, a plurality of parameters may be measured for each frame, and any combinations of parameters may be provided. Localization in a body lumen, such as the GI tract, may be determined, for example, as described in U.S. Pat. No. 5,604,531 and/or U.S. application Ser. No. 10/150,018, both assigned to the common assignee of the present application and which are hereby incorporated by reference. Examining local changes of parameters, such as temperature and pH for example, may provide additional information to, for example, a physician, for, for example, identification and localization of pathologies.

According to some embodiments of the present invention, the temperature sensitive material 20 may be connected to an in vivo device 10 or a part thereof, such as an in-vivo camera system. The in-vivo device 10 may be included on or within any suitable apparatus that may be introduced into the body to view the interior, such as an endoscope, a catheter, an ingestible capsule, and any other suitable imaging device. In-vivo imaging device may also be autonomous, such as in the case of an autonomous capsule. “Temperature-sensitive” in the context of the present invention may be defined as reactive to changes in temperature. This temperature change may include a range of temperatures or just a change from a reference temperature to another temperature. In other embodiments, device 10 may include pressure-sensitive, pH sensitive or alternative environmental parameter measuring color-changing materials. Thus, different properties within the environment of the body lumen can be measured in a similar manner to the one described for temperature hereinbelow. Other parameter measuring materials, which may not be color-changing, may be used for measuring in vivo environmental changes. For example, known pH and pressure sensors may be used for determining in vivo environmental changes.

As shown in FIG. 2, temperature-sensitive color-changing material 20 may be placed on the inside of device 10, the sensitive (color changing) portion facing inwards towards the device imager 46. By placing material 20 on the inside of device 10, many potential problems, such as complications associated with the biocompatibility and the resilience of material 20 in light of bodily fluids and pH changes etc., may be avoided. However, it should be apparent that color-changing material may also be placed on the outside of device 10. The attachment or placement of material 20 may be accomplished in a plurality of ways. For example, in one embodiment material 20 may be in the form of paint, and may be painted onto device 10. In another embodiment, material 20 may be attached onto device 10 with adhesive. In another embodiment, material 20 may be sprayed onto device 10 as a coating. In other embodiments material 20 may be temperature adhered (welded), or adhered using pointwise binding or any other suitable means. The color changing material may alternatively be attached to a substrate (either transparent or non-transparent), and the substrate may be attached to, for example, the envelope of device 10. The color changing material 20 may be of other forms and may be adhered to the in-vivo imaging device in other ways.

According to some embodiments of the present invention, light from at least one light source 43 may be directed towards and/or through temperature-sensitive color-changing material 20. Light source 43 may include one or more components, for example, light emitting diodes (LEDs), which may be placed in various locations within device 10. Light source 43 may additionally or alternatively be used as illumination source 42 (of FIG. 1), to illuminate the environment being imaged (outside of device 10). A separate illumination source 42 may be included for illumination material 20 and/or the in vivo environment. As local environmental changes, such as temperature changes, may cause color-changing material 20 to change color, an imager, such as 46, may acquire frames that capture the color of material 20 to determine its color at each selected point in time. For example, imager 46 may record or otherwise acquire an image of temperature sensitive color changing material 20 “n” times per second, thereby enabling the generation of data indicating the temperature in an in vivo environment for “n” intervals per second etc. In some embodiments, imager 46 may be a CMOS imager or any other suitable imager. Other light sources and/or imaging units may be used. Transmitter 41 may transmit at least the color changing data to data receiver unit 12.

In some embodiments of the present invention, device 10 includes at least one viewing window 21 through which the light from light source 43 and/or illumination source 42 (of FIG. 1) may illuminate inner portions of body lumen, such as the digestive system. Color-changing material 20, as described above, may be adhered or otherwise placed on the viewing window 21 in such a way that parts or all of material 20 remain transparent and preferably provide minimal or no viewing barriers or limitations to the imager's 46 view of the environment external to device 10. For example material 20 may be a spot or portion relative to the window 21. Thus, when device 10 is swallowed or otherwise enters into a body lumen, such as the gastrointestinal tract, and proceeds to travel through the length of the lumen, the imager 46 may image the lumen wall and/or environment while simultaneously imaging the color-changing material 20. In this way, any change of color, due to an environmental change such as a change in temperature in the GI environment, for example, may be visible in the images acquired by imager 46 from, for example, the GI tract. Such embodiments may provide the viewer, for example, with images for each frame acquired, or for temperature data superimposed on each acquired image. Such an image, which may be a shaded image or temperature data etc., may be located in a portion of an image. In other embodiments suitable pH, pressure and/or other environment sensitive material may be used.

In some embodiments, a temperature-sensitive color-changing material 20 that may be used may be a thermotropic liquid crystal (TLC) paint or coating etc., such as are offered by Hallcrest, Inc. of Glenview, Ill. Such TLCs, which may be cholesteric (comprised of sterol-derived chemicals), chiral nematic (comprised of non-sterol based chemicals) liquid crystals, a combination of the two, or any other forms or combinations of forms, may provide color changes in response to temperature changes. These color changes may be reversible or hysteretic. TLC may be used in various forms according to several embodiments of the present invention, including but not limited to paints, microencapsulated coatings and slurries, TLC coated polyester sheets, and unsealed films. Any other temperature-sensitive color-changing materials may be used, independently or in any combination.

In some embodiments of the present invention, temperature-sensitive color-changing material 20 may be sensitive to changes within a small range of temperatures, for fine, precise detection of temperature changes. For example, material 20 may be sensitive for small changes between, for example, 36 degrees Celsius and 39 degrees Celsius. In another embodiment, temperature-sensitive color-changing material 20 may be sensitive to changes within a larger range of temperatures, for example, from 30 degrees Celsius to 40 degrees Celsius, for more coarse determination of temperature and/or of temperature changes. Any range of sensitivities may be possible depending on the material used. For example, an in-vivo imaging device may be designed to determine temperature changes in the stomach, which typically range from 37-38 degrees Celsius, using a color changing material 20 that is highly sensitive to temperature change, such as a material that may change a shade of color for every 0.05 degree Celsius temperature change. However, during intake of cold or warm food, for example, temperature in the stomach may have larger range, for example 32-42 degrees Celsius, which may therefore require usage of color changing material 20 that is less sensitive to temperature change. In some embodiments, a combination of materials that may be sensitive to different temperature ranges etc. may be used so as to provide a required resolution and accuracy. It should be appreciated that any number of combinations of temperature-sensitive color-changing materials 20 may be used so as to optimize the temperature detecting capabilities of the system 100.

According to some embodiments of the present invention, in vivo measurements may be determined by processing, calibration, and/or calculation of acquired color information from color-changing materials, by computer image processing means. According to this embodiment, results of such processing and analyses may be presented to a user in various forms, such as graphs, charts, maps etc., in addition to color maps. Of course, other parameter sensitive materials may be used, and other displays of results may be generated.

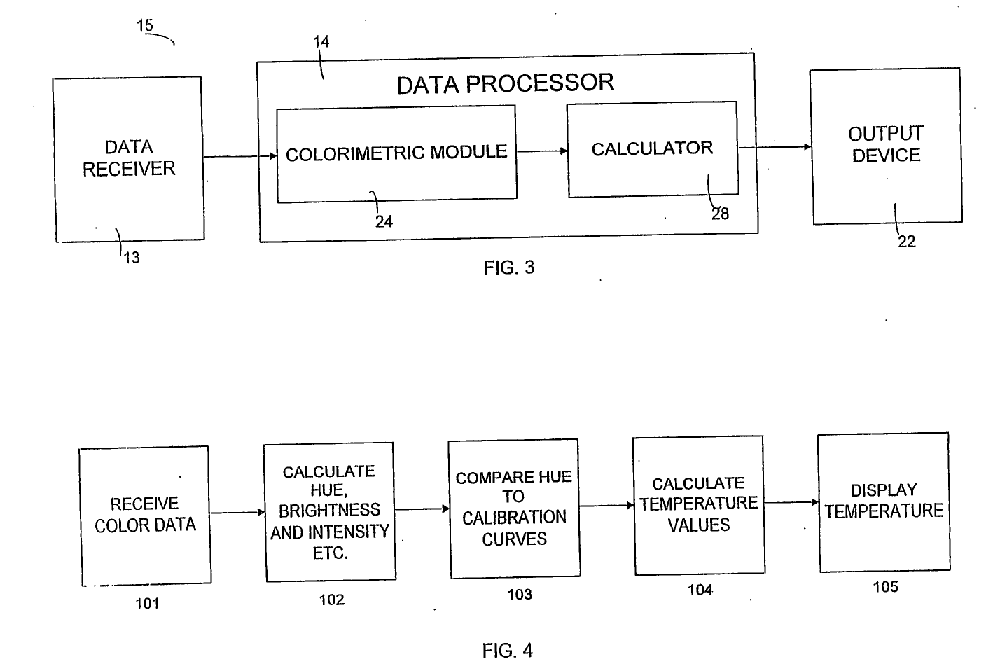

Reference is now made to FIG. 3, which illustrates a data flow diagram showing the steps of determining parameter changes, for example temperature changes, within the body. These changes may typically be based on the data of color images acquired from, for example, an environment parameter color-changing material or other environment-sensitive material. System 15 may comprise data receiver 12, data processor 14, and output device 22. Data processor 14 may comprise a calorimetric module 24, for determining color parameters, such as image color (hue), and a calculator 28, for calculating the temperature based on the calibration curve. According to one embodiment of the invention, data processor 14 may be a standard computer accelerator board, high performance computer, multiprocessor, microprocessor, or any other serial or parallel high performance processing machine. Data processor 14 may include or be part of, for example, a personal computer or workstation etc In one embodiment, processor 14 may be inside in-vivo imaging device 10. In another embodiment, processor 14 may be outside of in-vivo imaging device 10 at a remote location. Output device 22 may be a display monitor or any other unit for outputting analog and/or digital data, in the forms of audio, visual, and/or video signals etc. The data may be output, for example, as video data, graphs, tables, audio signals, color images, maps, charts or in any other suitable form. For example, output device 22 may be an indicator that produces signals upon environmental changes, or may be a monitor for displaying temperature maps superimposed over images etc. Output device 22 may output additional information such as image data. Other data flow components may be used.

Colorimetric module 24 may be used to analyze data received by the data receiver 12 from a plurality of data frames. This analysis may be used to determine color components of the data received. Colorimetric module 24 may also determine and express the color components in calibration curves according to hue, saturation and/or brightness etc. Calculator 28 may be used for comparing hue values derived by Colorimetric module 24 to previously obtained calibration curves, and to calculate temperature values for each frame of image data received by data receiver unit 12. These calculations may be used to determine the absolute values of the parameter as well as magnitude of changes between or across one or more image frames.

Reference is now made to FIG. 4, which illustrates an example of steps that may be implemented to determine environmental changes, for example temperature changes, within a body. These changes may typically be determined using colored images acquired from an environment parameter color-changing material. Steps of FIG. 4 may be accomplished using system 15 of FIG. 3, or any other suitable system. Data receiver 12 may receive (step 101) data, including image data, captured by, for example, in-vivo imaging device 10 of FIG. 2 or any other in-vivo imager.

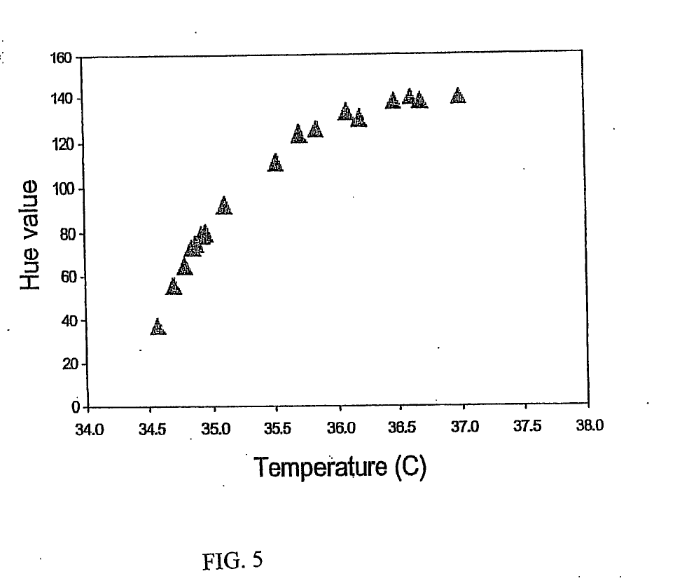

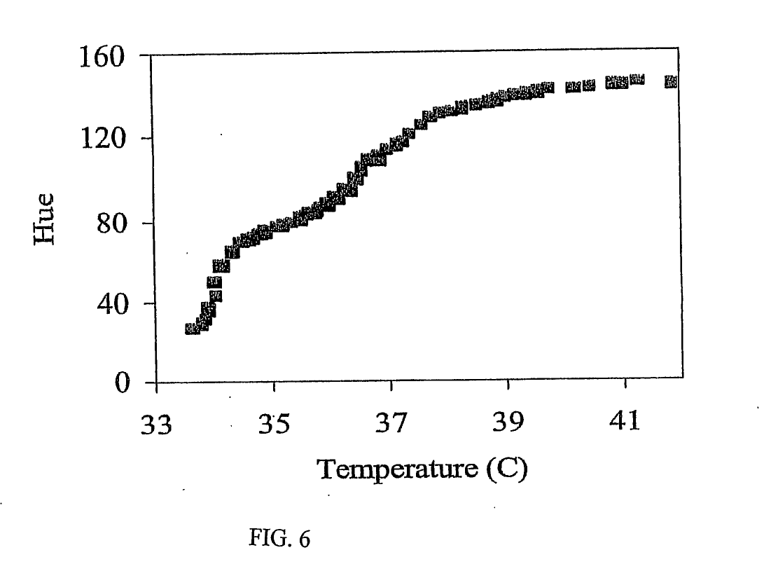

Data processor 14 may convert the received image data into parameter information, such as temperature information. Colorimetric module 24 may calculate (step 102) color components of the received data for each image frame. Colorimetric module 24 may also express the color components according to, for example, hue, saturation and/or brightness. Other color data formats may be used. Calculator 28 may locate (step 103) hue values on previously obtained calibration curves, as shown in FIGS. 5 and 6 and described more filly below. Calculator 28 may calculate (step 104) values (e.g. temperature values) for each frame of image data received by data receiver 12. Output device 22 may display (step 105) at least one parameter value, such as temperature, for each frame recoded by in vivo imaging device 10, for example, in the gastrointestinal tract. In some embodiments the data output by output device 22 may be, for example, a graph of parameter changes, such as temperature changes, over the length of the tract In other embodiments the display may be a graph of temperature changes over time. The display may also combine temperature changes over changes in space and changes in time. The display or output may also be in the form of alternative signals, including video signals, audio signals, etc. Any combination of the above steps may be implemented, and other steps or series of steps may be used. In alternate embodiments other units or combinations of units may perform such steps. Other steps or series of steps may be performed.

In other embodiments of the present invention, parameter mapping, such as temperature, pH and/Qr pressure mapping etc., may be accomplished using system 15 of FIG. 3. In such embodiments, parameter maps may be created. For example, image receiver 12 may receive image frames that include frame color data. A variety of frames may be combined to generate at least one chart of colors relating to parameter changes across multiple frames. The chart thus generated may be displayed in a pattern as to relate to or resemble the environment from where they were acquired. For example, if the temperature measured at one point differs from that at another point, a processor unit may determine the location, frame number, time, position etc. of each point in relation to other points, thereby generating a display of the differences in temperature at different locations within a body lumen, for example the GI tract. In such a case, for example, the color data from the various frames may be divided into a grid of pixels, for example, and the calculations described above for FIG. 4 may be done for each pixel, thereby generating an image map representing one or more environmental parameters, such as temperatures, at each point in the lumen where the images of material 20 were acquired. In this way the resulting parameter map may be displayed for a section, portion, or block etc. of an in-vivo environment, optionally at each point in time.

According to some embodiments of the present invention, additional environment parameter sensitive color-changing materials 20, or other material that may be non-color sensitive, may be added to an ingestible imaging device. For example, a plurality of color-changing materials may be added to the in-vivo imaging device 10, to obtain environment parameter information from a plurality of areas around the in-vivo imaging device simultaneously.

Reference is now made to FIG. 5, which is an example of a calibration curve optionally generated by the data processor 14, by comparing, for example, hue values to temperature values for a temperature-sensitive color-changing material. Such a calibration curve may represent a temperature-sensitive color-changing material, such as a temperature sensitive material that is moderately sensitive to changes in temperature. As shown in FIG. 5, the hue value changes over a range of temperatures from 34.5 to 37.5 degrees Celsius. Of course, other parameter sensitive material may be used, independently or in combination. Other values may be used in quantifying the in vivo changes reflected by the color changing material.

Reference is now made to FIG. 6, which is an example of a calibration curve optionally generated by the data processor 14, by comparing hue values to temperature values for a temperature-sensitive color-changing material. Such a calibration curve may represent a temperature-sensitive color-changing material that is relatively insensitive to changes in temperature (as compared to the material described in FIG. 5 above). As shown in FIG. 6, the hue value changes over a range of temperatures from 33-42 degrees Celsius. One or more color-changing materials with varying sensitivities to environment parameters may be used, alone or in combination, depending on the precision and resolution of the data output required. Other values may be used in quantifying the in vivo changes reflected by the color changing material.

It should be noted that calculation of environmental parameters, such as temperature measurements, may be done simultaneously with imaging of the tract, or it may be done at a later time. When done simultaneously, any areas of interest may be extracted from the image for further investigation.

It will be appreciated by persons skilled in the art that embodiments of the present invention may include pressure-sensitive, pH sensitive, or any other color-changing materials that are sensitive to changes in the environment. Other embodiments may include non-color-changing materials that are sensitive to changes in the environment. Any number of environment-sensitive materials may be used, individually or in any combinations. While the present invention has been described with respect to color-changing materials that are sensitive to environmental changes such as temperature, pressure and pH levels, the scope of the present invention may include usage of other materials that may be sensitive to temperature, pressure, pH or any other environmental parameters, and may indicate their sensitivity to environmental changes by changing their outputs in ways that are unrelated to color changes. The foregoing description of the embodiments of the invention has been presented for the purposes of illustration and description. It is not intended to be exhaustive or to limit the present invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the present invention.

1. A system enabling in vivo environment sensing, comprising:
an in-vivo imaging device;
at least one environment sensitive color-changing material; and
at least one data transmitter.

2. The system of claim 1, wherein said in-vivo imaging device is at least one device selected from the group consisting of swallowable capsules, endoscopes, and catheters.

3. The system of claim 1, wherein said material is adhered to the device according to one or more ways selected from the group consisting of painting, adhering, welding, point-wise binding and spraying.

4. The system of claim 1, wherein said material is one or more materials selected from the group consisting of thermotropic color-changing liquid crystal, litmus paper, shear sensitive liquid crystal, and color changing irreversible material.

5. The system of claim 1, comprising a plurality of color-changing materials, to sense a plurality of environmental parameter changes.

6. The system of claim 5, wherein said environmental parameter changes include one or more parameters selected from the group consisting of in vivo temperature, in vivo pressure, in vivo pH level, and in-vivo bacteria level.

7. The system of claim 1, comprising:
at least one light source; and
at least one imager.

8. The system of claim 1, comprising:
at least one data receiver unit;
at least one data processing unit; and
at least one data output unit.

9. The system of claim 8, wherein said data processor unit comprises:
at least one colorimetric module; and
at least one calculator.

10. A system enabling in vivo sensing, comprising:
an in-vivo imaging device;
at least one environment sensitive color-changing material;
at least one data receiver unit;
at least one data processing unit; and
at least one data output unit.

11. The system of claim 10, wherein said data processing unit comprises:
at least one calorimetric module; and
at least one calculator.

12. A system enabling in vivo sensing, comprising:
means for indicating a change of at least one environmental parameter;
means for lighting said indicating means; and
means for acquiring in vivo images, said images including environmental parameter changes indicated by said indicating means.

13. The system of claim 12, wherein said means for indicating changes in at least one environmental parameter is a color changing material.

14. A method enabling in vivo sensing, comprising:
radiating light onto at least one color changing environment sensitive material coupled to an in-vivo imaging device;
acquiring a plurality of images from said material; and
transmitting said acquired images to a data receiving unit.

15. The method of claim 14, comprising:
receiving color change data from said in-vivo imaging device; and
calculating at least one parameter value from said color change data.

16. The method of claim 14, comprising processing said acquired data by a data processing unit.

17. The method of claim 16, wherein said processing said acquired data comprises:
calculating the color components of received data for each image received; and
comparing color component values for each image to at least one calibration.

18. The method of claim 16, wherein said processing said acquired data comprises:
calculating the color components of received data for each image received;
expressing the color components of each image according to at least one of hue, saturation and brightness;
comparing color component values for each image to at least one calibration curve generated from previous images; and
calculating values for each image received by said data receiving unit.

19. The method of claim 14, comprising outputting data relating to said environment parameter changes.

20. The method of claim 14, wherein said environment sensitive material is one or more color changing materials selected from the group consisting of temperature sensitive material, pressure sensitive material, pH sensitive material and bacteria sensitive material.

21. The method of claim 14, comprising generating a value for at least one environmental parameter for each image, and displaying said value for each image of acquired data.

22. The method of claim 21, comprising:
generating at least one environment parameter value for one or more acquired images; and
displaying at least one environment parameter map for one or more acquired images.

23. The method of claim 22, wherein said generation of at least one environment parameter map comprises:
receiving a plurality of image frames;
combining a plurality of frames to generate at least one image including colors relating to parameter changes across said plurality of frames;
generating at least one image representing an environment;
dividing said color image into a grid of pixels, each pixel relating to at least one image frame received;
calculating a color for each said pixel, according to image frames received; and
displaying said pixels as a map of parameter changes for said environment.

24. The method of claim 23, comprising displaying said map of parameters at each selected point in time.

25. A method enabling in vivo sensing, comprising:
receiving image data from an in-vivo imaging device, said device having at least one color changing environment sensitive material; and
outputting image-related data.

26. The method of claim 25, comprising:
receiving image data from a plurality of frames;
processing image data for each frame; and
comparing image data from two or more frames.

27. A method enabling in vivo sensing, comprising:
placing at least one environment sensitive material to an in-vivo imaging device;
sensing environmental parameter changes by said environment sensitive material;
acquiring data indicating said environment parameter changes from said environment sensitive material;
transmitting said acquired data to a data processing unit;
processing said acquired data by said data processing unit; and
outputting data relating to said environment parameter changes.

28. The method of claim 27, wherein said environment sensitive material is a color changing material.

29. A system enabling determining of in vivo environmental conditions, comprising:
a data receiver unit, to receive at least image data related to an environment sensitive color-changing material; and
a processing unit to process said data, thereby determining the in vivo environmental conditions.

30. The system of claim 29, comprising at least one output device.

31. The system of claim 29, comprising at least one storage unit.

32. The system of claim 29, wherein said processing unit comprises:
at least one colorimetric module; and
at least one calculator.

33. The system of claim 29, wherein said in vivo environmental conditions include one or more parameters selected from the group consisting of in vivo temperature, in vivo pressure, in vivo pH level, and in-vivo bacteria level.

34. A system enabling determining changes in vivo environmental conditions, comprising:
a data receiver unit, to receive at least data related to an environment sensitive color-changing material;
a processing unit to process said data, thereby calculating in vivo environmental changes; and
at least one data output device.

35. The system of claim 34, wherein said data comprises a plurality of frames, each of said frames including data of color status of said environmental sensitive color-changing material.

36. A system enabling determining of changes in vivo environmental conditions, comprising:
data receiving means for receiving at least data related to an environmental sensitive color-changing material;
data processing means for processing said data, thereby calculating changes in the in vivo environment; and
data output means for displaying data related to the in vivo environment.

37. The system of claim 36, wherein said processing means comprise:
colorimetric calculation means for determining color components of said data related to said environmental sensitive color-changing material;
calculator means for comparing hue values derived by said colorimetric calculation means to at least one calibration curve, and calculating environment parameter values for each data frame received by said data receiving means.
